(19) United States
(12) Patent Application Publication
(10) Pub. No.: US 2011/0270093 A1
(43) Pub. Date: Nov. 3, 2011

(54) OPTICAL EXAMINATION DEVICE ADAPTED TO BE AT LEAST PARTIALLY INSERTED INTO A TURBID MEDIUM

(75) Inventors: Adrien Emmanuel Desjardins, Eindhoven (CA); Martinus Bernardus Van Der Mark, Eindhoven (NL); Bernardus Hendrikus Wilhelmus Hendriks, Eindhoven (NL); Gert 'T Hooft, Eindhoven (NL)

(73) Assignee: KONINKLIJKE PHILIPS ELECTRONICS N.V., EINDHOVEN (NL)

(21) Appl. No.: 13/142,485

(22) PCT Filed: Jan. 18, 2010
(86) PCT No.: PCT/IB2010/050208

§ 371 Date: Jul. 18, 2011

(30) Foreign Application Priority Data
Jan. 23, 2009 (EP) 09151274.9
Nov. 3, 2009 (EP) 09174834.3

(51) Int. Cl. A61B 6/00; H01L 27/00; G01J 3/00; H01J 5/02; G01J 1/42

(52) U.S. Cl. 600/476; 250/239; 250/208.2; 356/300; 250/208.1

(57) ABSTRACT

An optical examination device (10) adapted to be at least partially inserted into a turbid medium is provided. The optical examination device comprises a shaft portion (21) adapted to be inserted into the turbid medium, the shaft portion (21) comprising a tip portion (22) adapted to be the foremost portion during insertion into the turbid medium. At least one light source device adapted to emit abeam (11) of broad-band light is provided in the region of the tip portion (21). The beam (11) of broad-band light comprises different wavelength bands (2a, 2b, . . . , 2n) which are differently modulated. At least one photodetector (27a, 27b, 27c) for detecting broad-band light is provided in a region adapted to be inserted into the turbid medium of the shaft portion (21).

### FIELD OF INVENTION

The present invention relates to an optical examination device adapted to be at least partially inserted into a turbid medium.

### BACKGROUND OF THE INVENTION

In the context of the present application, the term light is to be understood to mean non-ionizing electromagnetic radiation, in particular with wavelengths in the range between 400 nm and 1400 nm. The term photodetector means a device which is capable of receiving incoming light and outputting an electric signal corresponding to the received light in response. The term turbid medium is to be understood to mean a substance consisting of a material having a high light scattering coefficient, such as for instance intralipid solution or biological tissue.

In many medical contexts, biopsies are the only method for confirming medical diagnoses. Needle biopsies are also known as fine needle aspiration cytology (FNAC), fine needle aspiration biopsy (FNAB) or fine needle aspiration (FNA). Such needle biopsies are employed to extract small amounts of tissue from a turbid medium which is formed by a mammal body, i.e. a human body or an animal body, for further analysis of the extracted tissue outside the body, e.g. by a pathologist under a microscope. Needle aspiration biopsies are frequently used for, below others, examining female breasts, prostates, lungs, thyroid, and bone. Compared to surgical biopsies, needle aspiration biopsies are less invasive, less expensive, less time-consuming, and come along with shorter recovery times of the patients being subject to the biopsy. For example, approximately one million needle biopsies are performed in the United States of America each year for the diagnosis of breast cancer.

Nowadays, tissue biopsies for taking tissue samples from the interior of a mammal body are performed without feedback from the biopsy needle. As a result, physicians lack information about the microstructure and the molecular composition of the tissue which is located immediately in front of the needle tip. As a result, there are often uncertainties about the location of the needle tip with regard to the tissue region from which sampling is desired.

In order to overcome this problem, in the absence of direct feedback from the biopsy needle, it is known to employ a variety of different imaging modalities to assist in needle positioning. Such imaging modalities include X-ray imaging, MRI (magnetic resonance imaging), and ultrasound imaging. Whilst these modalities are capable of providing useful information about the absolute location of the biopsy needle, the required information about the relative location of the biopsy needle with respect to the tissue (which is of particular interest) often cannot be achieved. The achieved spatial resolution is often inadequate for identifying small pathological masses. Further, the applied imaging modalities often show inadequate soft-tissue contrast for discrimination between benign and malignant tissues. A further common problem is that the applied imaging modalities often provide inadequate contrast for identifying small blood vessels or nerves which are in the path of the biopsy needle.

Due to these drawbacks, there are many cases in which blood vessels or nerves are inadvertently punctured during needle biopsy. Puncturing vessels with biopsy needles can be harmful to the patient, as internal bleeding may arise. Furthermore, puncturing nerves can also be particularly harmful to the patient. In view of this, it is not only important to acquire information with regard to the tissue which is located in front of the beveled part of the tip (i.e. in the area from which tissue can be extracted with the biopsy needle) but also to acquire information with regard to the tissue which is located in front of the foremost part of the needle tip (i.e. the tissue which will be punctured if the biopsy needle is moved farther forward).

The possibility of providing direct feedback from the biopsy needle via optical fibers exists. For example, optical fibers can be used to provide information about the tissue surrounding the needle tip. It is known that tissues can be differentiated by their respective optical absorption spectra (cf. for instance Zonios et al., “Diffuse reflectance spectroscopy of human adenomatous colon polyps in vivo”, Appl. Opt. 38(31), 1999, 6628-6637). In particular, the hemoglobin which is present in blood provides pronounced optical signatures.

In view of the above, it would be advantageous to detect light at the sides of a biopsy needle. For example, this would allow sensing light which has traveled around the sharp tip of the biopsy needle starting from the beveled side of the needle tip and arriving at the needle shaft. It is in principle possible to guide light to the tip of a biopsy needle via an optical fiber and emit the light to the tissue in front of the sharp tip of the biopsy needle. Further, it is possible to collect the light which has scattered in the region of tissue in front of the tip of the biopsy needle by means of one or more other optical fibers the ends of which are located in the region of the shaft of the biopsy needle. The optical fibers could, for instance, be integrated into the shaft of the biopsy needle. However, such a system comprises the following drawbacks: The required multimode fibers for collecting the scattered light typically comprise numerical apertures in the range of 0.2. This results in that only a small amount of the light which is incident on the surface at the end of the optical fiber can be collected. Further, the construction and manufacture of biopsy needles comprising a plurality of optical fibers is expensive. In order to perform spectroscopy with such a system, i.e. to acquire the distribution of a large number of different wavelengths or wavelength bands in the scattered light for each detection position which is formed by the end of a respective optical fiber, the collected light would have to be analyzed by a spectrometer specifically adapted for small intensities. In this case, acquisition of spectra for several detection positions would require a considerable amount of time.

### SUMMARY OF THE INVENTION

It is an object of the present invention to provide an optical examination device adapted to be at least partially inserted into a turbid medium which allows spectral analysis of a region of the turbid medium located in front of a tip portion more reliably, at lower costs, and with reduced data acquisition time.

This object is solved by an optical examination device adapted to be at least partially inserted into a turbid medium according to claim 1. The optical examination device comprises: a shaft portion adapted to be inserted into the turbid medium. The shaft portion comprises a tip portion adapted to be the foremost portion during insertion into the turbid medium. At least one light source device adapted to emit a beam of broad-band light is provided in a region adapted to be inserted into the turbid medium of the shaft portion. The beam of broad-band light comprises different wavelength bands which are differently modulated. At least one photodetector for detecting broad-band light is provided in the region adapted to be inserted into the turbid medium of the shaft portion. Since the optical examination device is provided with the at least one light source in the region of the shank portion which is adapted to be inserted into the turbid medium, the beam of broad-band light can be reliably emitted towards and scattered in the region of interest of the turbid medium, such as tissue located at a specific position in the case of medical applications. Since the beam of broad-band light comprises different wavelength bands which are differently modulated, spectroscopic information can be acquired with a simple photodetector in combination with a demodulation unit. The demodulation unit can be realized as a compact electronic circuit or can be implemented in software on a suitable processor. Thus, sophisticated and expensive spectrometers can be dispensed with. In this context, broad-band light comprising different wavelength bands means light which comprises a large number of wavelengths with continuous wavelength spectra in at least one wavelength band. Broad-band means that a wide range of wavelengths is covered. The plurality of wavelength bands can be modulated at different frequencies and/or timing sequences. Since the at least one photodetector is provided in the region which is adapted to be inserted into the turbid medium, scattered light can directly be detected inside the turbid medium with the at least one photodetector. Thus, the scattered light does not have to be coupled into optical fibers which would lead to the problem of only small numerical apertures available. Further, in a case in which a plurality of detection positions is provided, instead of an additional optical fiber for each detection position (which would be required if the scattered light would have to be guided to a spectrometer located outside the turbid medium such as a mammal body) only electrical connections from the photodetectors to the outside of the turbid medium (e.g. to the outside of the mammal body) are required. This comes along with a considerable cost reduction and results in a less complicated system. In particular, the at least one photodetector (or a plurality of photodetectors) can be arranged in a side region of the shaft portion.

If the at least one photodetector is electrically connected to a portion of the optical examination device adapted to remain outside the turbid medium, the spectroscopic information contained in a signal from the at least one photodetector can be conveniently analyzed outside the turbid medium. In the preferred case in which a plurality of photodetectors is provided at different positions of the shaft portion, all these photodetectors can preferably be electrically connected to the outside of the turbid medium.

According to one aspect, the at least one photodetector is a photodiode. Photodiodes can be conveniently fabricated with high detection efficiency and at low costs. Further, they can be realized in a very compact fashion such that integration into the shaft portion, compact arrangement on an inner or outer surface of the shaft portion, or compact arrangement on a core element to be placed in a hollow channel inside the shaft portion (such as a mandrin in the case of a biopsy needle) is possible.

According to an aspect, the shaft portion is provided with a plurality of photodetectors arranged at different positions relative to the shaft portion. In this case, spectroscopic information contained in the scattered light can be acquired at different spatial positions. As a consequence, spatial resolution of the properties of the region of the turbid medium (e.g. tissue) which is located in front of the tip portion becomes possible.

According to an aspect, the optical examination device comprises a demodulation and analysis unit adapted to perform a spectral analysis of a signal received from the at least one photodetector. In this case, information about the region of the turbid medium in front of the tip portion is analyzed with regard to the distribution of different wavelength bands. As a consequence, information about the scattering properties and/or chromophore concentration in this region of the turbid medium can be reliably acquired.

According to an aspect, the demodulation and analysis unit is adapted to perform spectral analysis of signals from a plurality of photodetectors and additionally exploit information about respective positions of the plurality of photodetectors. In this case, spatially resolved spectroscopic information becomes available which allows reconstructing two- or more-dimensional images of the region of interest of the turbid medium, in particular in front of the tip portion.

According to an aspect, the demodulation and analysis unit is adapted to reconstruct a multi-dimensional image of a region of interest of the turbid medium, e.g. a region which is located in front of the tip portion. In this case, the acquired information about the region of the turbid medium is conveniently visualized. The image can for instance be a two-dimensional or three-dimensional image. However, four-dimensional or higher dimensional images can also be realized, e.g. by using a color scale to represent a fourth dimension. The image can for instance represent the absorption and/or scattering coefficients in a spatially resolved manner or the spatially resolved distribution of one or more chromophores.

According to an aspect, the shaft portion forms at least a part of a biopsy needle. In this case, inadvertently puncturing tissue which should not be punctured, such as nerves or blood vessels, can be prevented. In an alternative, the shaft portion forms at least a part of a catheter or of an endoscope.

According to an aspect, the at least one light source device is formed by the end of a light guiding structure connected to a light generating unit adapted to provide the beam of broad-band light. In this case, the beam of spectrally coded broad-band light can be generated outside the turbid medium (e.g. outside a mammal body) and conveniently be guided to the tip portion via the light guiding structure. Thus, generation of the beam of spectrally coded broad-band light can be realized with high accuracy. The light guiding structure can for instance be arranged in the material of the shaft portion or be provided in a core element adapted to be placed in a hollow channel inside the shaft portion (such as a mandrin in the case of a biopsy needle). For example, the light guiding structure can be formed by a light guiding fiber (optical fiber).

According to an aspect, the at least one photodetector is embedded in the material of the shaft portion, preferably such that it does not protrude from the shaft portion. In this case, the provision of the at least one photodetector does not negatively affect insertion of the shaft portion into the turbid medium which is particularly relevant if the turbid medium is formed by a living mammal body.

According to an aspect, the optical examination device is adapted such that a high-frequency modulation in a frequency range above 50 MHz is imposed on the beam of broad-band light. This high-frequency modulation is imposed on the beam in addition to the specific modulation for different wavelength bands. The high-frequency modulation can be utilized to extract additional optical properties from the tissue in front of the tip, such as optical scattering coefficients or fluorescence lifetime coefficients (in the case that natural fluorescence or fluorescence of contrast agents is exploited).

According to one aspect, the optical examination device is a medical device adapted to be at least partially inserted into a mammal body. In this case, the shaft portion is adapted to be inserted into the mammal body and the at least one photodetector is arranged in a region which is adapted to be inserted into the mammal body of the shaft portion.

If the at least one light source device is provided in the region of the tip portion, the beam of broad-band light can be reliably emitted towards and scattered in the region of the turbid medium which is located in front of the foremost tip, such as tissue in the case of medical applications.

### BRIEF DESCRIPTION OF THE DRAWINGS

Further features and advantages of the present invention will arise from the detailed description of embodiments with reference to the enclosed drawings.

FIG. 1 schematically shows an optical examination device according to a first embodiment.

FIG. 2 schematically shows a foremost part of a shaft portion of the optical examination device.

FIG. 3 schematically shows the shaft portion of FIG. 2 with a core element inserted.

FIG. 4 schematically shows a light generating unit.

### DETAILED DESCRIPTION OF EMBODIMENTS

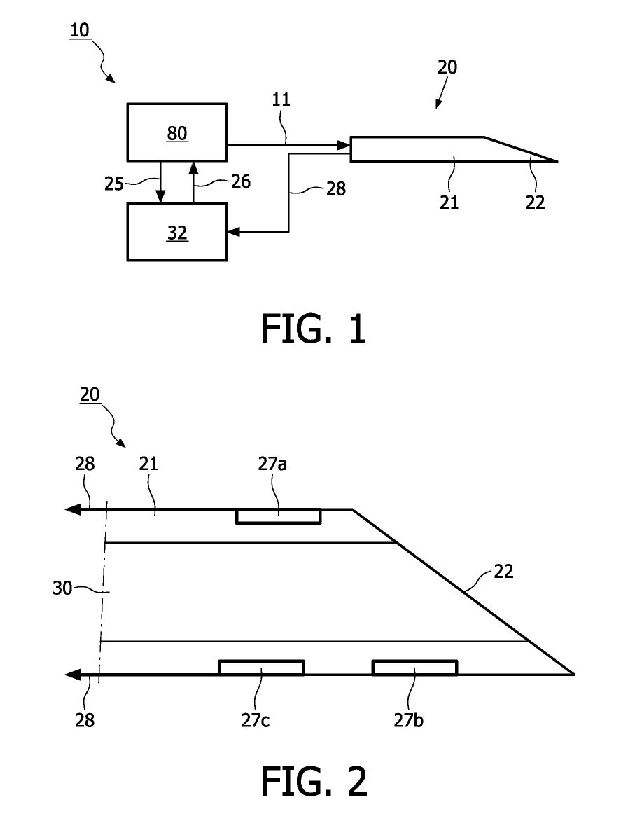

An embodiment of the present invention will now be described with reference to FIGS. 1 to 4. The optical examination device 10 comprises a part 20 adapted to be inserted into a turbid medium. The optical examination device 10 which will be described with reference to the figures as an exemplary embodiment is formed by medical device and, in this case, the part 20 is adapted to be inserted into a mammal body (i.e. a human or animal body). In this case, the turbid medium is formed by the mammal body. In the exemplary embodiment which will be described with reference to the Figures, the part 20 is formed by a biopsy needle. The part 20 has a shaft portion 21 which comprises a tip portion 22. During insertion into the turbid medium, the tip portion 22 forms the foremost portion of the shaft portion 21. The shaft portion 21 has a tubular shape with a substantially circular cross-section and comprises a beveled shape in the region of the tip portion 22. The shaft portion 21 is provided with a hollow channel 30 which in the depicted example of a biopsy needle serves for extracting tissue samples from a mammal body. The shaft portion 21 is adapted such that the hollow channel 30 can be filled by a core element 31 which can be arranged in the hollow channel 30. The core element 31 can be retracted from the hollow channel 30 once the tip portion 22 is located at the position from which a tissue sample is to be taken. In the described case of a biopsy needle, the core element 31 is formed by a mandrin.

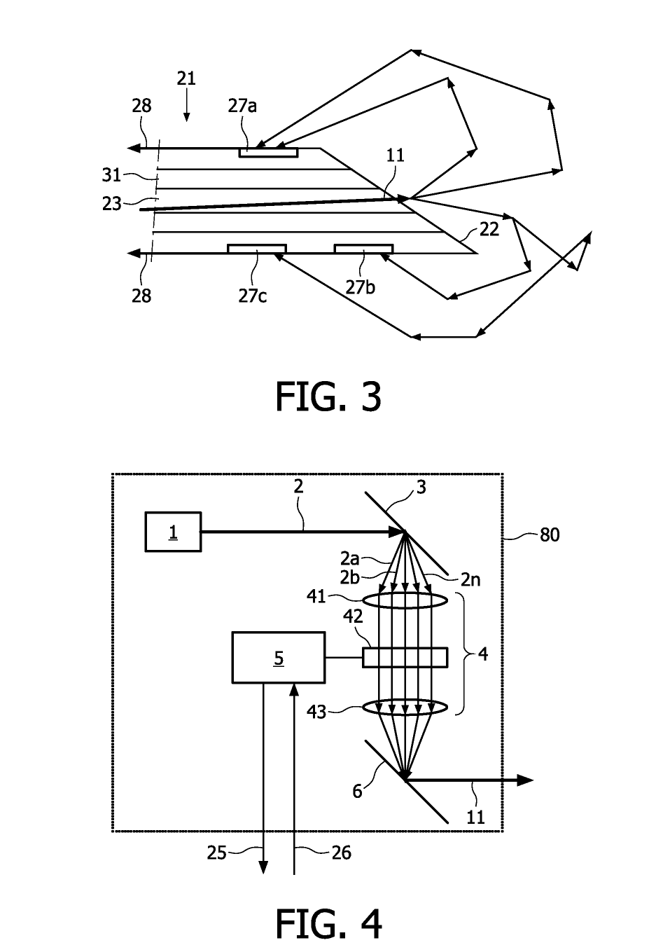

FIG. 2 shows the shaft portion 21 without core element 31 placed in the hollow channel 30. FIG. 3 shows the shaft portion 21 with inserted core element 31. The part 20 is connected to a light generating unit 80 which will be explained in more detail below. The light generating unit 80 provides a beam 11 of broad-band light comprising different wavelength bands which are differently modulated. In the exemplary embodiment, the beam 11 is guided to the tip portion 22 via a light guiding structure 23 which in the example is formed by an optical fiber. In the example given here, the light guiding structure 23 is centrally arranged in the core element 31. One end of the light guiding structure 23 which is positioned in the region of the tip portion 22 is adapted such that the beam 11 of broad-band light can be emitted to tissue which is located in front of the tip portion 22 (in a direction in which the shaft portion is inserted into the turbid medium such as a mammal body). Thus, the optical examination device 10 is adapted such that the beam 11 of broad-band light can be emitted to the region of the turbid medium (e.g. tissue) in front of the tip portion 22 such that the light is scattered in this region.

Further, at least one photodetector for detecting broad-band light is provided in a region of the shaft portion 21 which is close to the tip portion 22, in particular at the side of the shaft portion 21. In the exemplary embodiment shown in the Figures, three photodetectors 27a, 27b, and 27c are provided on the shaft portion 21, in particular embedded in the material of the shaft portion 21 such that they do not protrude from the shaft portion 21. It should be noted, that the number of photodetectors is not limited to this example and other numbers of photodetectors (even high numbers) may be provided. Further, as will become apparent from the following description, the provision of only one photodetector is also possible. The photodetectors 27a, 27b, 27c can for instance be formed by photodiodes. The photodetectors 27a, 27b, 27c are connected to a demodulation and analysis unit 32 via respective electrical connections 28. The demodulation and analysis unit 32 can e.g. be formed by a computer which is adapted accordingly. In the region of the shaft portion 21, the electrical connections 28 can for instance be arranged on the outer surface of the shaft portion 21. In this case, they are preferably protected from damage by a protective coating. Such a protective coating can also be used to insulate electrical connections. Alternatively, the electrical connections 28 can also be embedded in the material of the shaft portion 21 or arranged in the hollow channel 30.

The light generating unit 80 will now be described with reference to FIG. 4. The light generating unit 80 comprises a light source 1 emitting a collimated beam 2 of broad-band light, a band separator 3, a spatial light modulator 4, and a light recombining unit 6.

The light source 1 is chosen such that white light with high power and brightness is emitted. In this context, white light means that the light has a broad optical wavelength bandwidth which is sufficient for supporting the intended measurement. I.e. the beam 2 comprises a continuous broad band of wavelengths covering a large plurality of wavelengths, preferably in the visible, IR, and/or NIR. The light source 1 may be pulsed. For example, the light source 1 is an extremely bright white light source based on super-continuum generation. For example, this is achieved by using intense femto-second light pulses propagating through a holey fiber. However, it is also possible to use a rather simple lamp emitting white light. The broad bandwidth of the beam 2 allows for a large number of spectral points to be acquired, as will become apparent in the following. In this context, the term “spectral points” is used for measured signals at different wavelengths or frequencies, respectively. Thus, a large number of spectral points correspond to a large number of data for different wavelengths or frequencies, respectively.

The collimated beam 2 of broad-band light is directed to the band separator 3. The band separator is adapted such that it spatially separates a plurality of wavelength bands (2a, 2b, . . . , 2n) contained in the beam 2 of broad-band light. For example, the band separator 3 can be formed by a grating adapted for spatially splitting different bands of wavelengths contained in the beam 2 of broad-band light. However, it can also be formed by another kind of wavelength dispersive element such as a prism, for example. It should be noted that the different bands of wavelengths neither need to have the same width with respect to wavelength range nor the same wavelength spacing with respect to each other (wavelength spacing).

The spatially separated wavelength bands (2a, . . . , 2n) are directed to the spatial light modulator (SLM) 4 for spatially modulating the separated wavelength bands in such a way that each of the wavelength bands (2a, . . . , 2n) receives a specific modulation. In the present embodiment, the spatial light modulator 4 is of the transmission-type. However, spatial light modulation can also be realized in a reflection type arrangement. The spatial light modulator 4 comprises an input lens 41, a light modulating unit 42, an output lens 43, and a modulation source 5. The input lens 41 makes the respective beams of the distinct wavelength bands parallel. The light modulating unit 42 is connected to the modulation source 5 which controls the operation of the light modulating unit 42. The light modulating unit 42 can be mechanically realized, e.g. in form of a dedicated Nipkow-type disk or chopper or rotating polygon or the like. Preferably, the light modulating unit 42 is formed by a micro-mirror device or a liquid crystal device. Also the combination of any of these elements put in series in the light path is possible. For example, one element providing fast repetitive (periodic) modulation and another element providing a slowly varying adjustment of intensity can be provided.

Different ways of light modulation which are known in the art can be applied. For example frequency division multiplexing can be applied or time division multiplexing or both. The modulation scheme according to which modulation of the wavelength bands (channels) is performed is given by the light modulating unit 42 cooperating with the modulation source 5.

The independently modulated wavelength bands (2a, 2b, . . . , 2n) are recombined to a collimated beam 11 of spectrally encoded broad-band light by a light recombining unit 6 which may e.g. be formed by another grating or other wavelength dispersive element. In the embodiment, the band separator 3, the light recombining unit 6, the lenses and the light modulating unit 42 are arranged in a so-called 4-f configuration. However, the invention is not restricted to such an arrangement.

The collimated beam 11 of spectrally encoded broad-band light is then guided to the tip portion 22 of the shank portion 21 as has been described above. In the exemplary embodiment, the beam 11 of spectrally encoded broad-band light is coupled into the light guiding structure 23 in the light generating unit 80.

Operation of the optical examination device 10 will now be described. As been described above, when the shaft portion 21 has been inserted into a turbid medium, the beam 11 of spectrally encoded broad-band light is emitted towards the region of the turbid medium which is located in front of the tip portion 22. Due to the turbid nature of the turbid medium, the light is multiply scattered in the region of the turbid medium which is located in front of the tip portion 22 (as schematically indicated by a plurality of arrows in FIG. 3. A part of the light which has been scattered will be incident on the photodetectors 27a, 27b, and 27c. In response to the incident light, the photodetectors 27a, 27b, and 27c each generate an electric signal corresponding to the incident light. These electric signals are transmitted to the demodulation and analysis unit 32 via the electrical connections 28. Due to the beam 11 used for illuminating the turbid medium being spectrally encoded as has been described above, spectroscopic information can be analyzed based on the electric signals from the photodetectors 27a, 27b, and 27c.

In the demodulation and analysis unit 32, the signals detected by the photodetectors 27a, 27b, 27c are decoded/demodulated by a demodulation unit in order to restore the spectroscopic information contained in the diffuse light emanating from the turbid medium at the respective positions of the photodetectors 27a, 27b, and 27c. In order to allow reliable demodulation, the demodulation and analysis unit 32 is provided with a modulation signal 25 from the modulation source 5 in the light generating unit 80. The modulation signal 25 indicates the performed modulation. The modulation signal 25 allows the demodulation and analysis unit 32 to perform the appropriate demodulation operation. The demodulation unit of the demodulation and analysis unit 32 can for example be realized as a relatively cost-efficient and compact electronic circuit. Alternatively, it can be implemented in software running on a digital processor in the demodulation and analysis unit 32. In any case, the medium-specific optical spectra as imprinted by the turbid medium on the light incident on the respective photodetectors 27a, 27b, and 27c can be obtained corresponding to the different detection positions with high detection efficiency. It should be noted that, due to the above described spectrally encoding of the different wavelength bands, spectroscopic information can be acquired for each photodetector by way of the demodulation process. The demodulation and analysis unit 32 analyses the frequency content in the signal from the respective photodetector 27a, 27b, or 27c to determine the optical spectrum. Thus, intensity distributions of the respective wavelength bands can be determined from the electrical signals from the photodetectors 27a, 27b, and 27c. Thus, the described optical examination device 10 allows spectroscopy without requiring expensive and bulky spectrometers.

Further, the demodulation and analysis unit 32 can exploit information about the spatial position of the different photodetectors 27a, 27b, and 27c and evaluate the different intensity distributions of the light over the photo detectors.

In the exemplary embodiment, the demodulation and analysis unit 32 is adapted to process the signals corresponding to the different photodetectors 27a, 27b, and 27c using the principles of optical tomography in order to reconstruct images of the turbid medium in the region of the tip portion 22 from the provided spectroscopic information. The demodulation and analysis unit 32 can exploit a number of different reconstruction algorithms known in the art in order to reconstruct at least one image of properties of the turbid medium. Thus, the combination of spectroscopic and spatial information can e.g. be used to differentiate anatomical structures. For example, blood vessels can be distinguished from nerves. Different anatomical structures can be identified even if they are located several millimeters ahead of the needle tip.

Thus, according to the embodiment, a number of predefined wavelength bands (channels), which may have different width and or spacing, from a collimated white light source can each be coded in frequency domain and time domain using the band separator 3 and the spatial light modulator 4 (SLM). The wavelength bands are recombined to a single collimated beam 11 by a light recombining unit 6. The collimated and encoded beam 11 of possibly arbitrarily large optical bandwidth (white light) is used to illuminate the region of the turbid medium in front of the tip portion. According to the embodiment, the diffuse light emanating from the turbid medium is detected by a plurality of photodetectors 27a, 27b, and 27c. Respective signals from the photodetectors are demodulated such that optical spectra at different detection positions are obtained with high detection efficiency. The respective received signals are decoded/demodulated for each detection position to restore the spectroscopic information and hence obtain the medium-specific optical spectra as imprinted by the turbid medium on the light emanating from the turbid medium.

It is possible to operate the spatial light modulator 4 such that the different wavelength bands are modulated in a non-sinusoidal fashion, for example using square waves.

It is further possible to operate the spatial light modulator 4 such that a complex modulation scheme is followed in which adjacent channels (wavelength bands) are not adjacent in the translated RF domain on the detection side. In this case, the relevant channels are independently modulated such that, for the demodulation and analysis unit 32 demodulating the signals corresponding to the diffuse light detected at the detection positions, these relevant channels are not located adjacent to each other.

In the exemplary embodiment shown in the Figures, a feedback signal 26 from the demodulation and analysis unit 32 to the modulation source 5 in the light generation unit 80 is provided. With this feedback signal 26, the encoding scheme used for the broadband light can be modified dynamically in dependence on the electrical signals from the at least one photodetector 27a, 27b, 27c. For example, the order and/or distribution of the wavelength bands may be changed between measurements and the joint results of the different measurements can be taken to identify and suppress effects of cross-talk. For example, an a priori known feature in the spectrum may mask another, more subtle but important feature in one configuration but not in another configuration of channel order and/or distribution. Thus, if the order and/or distribution of the wavelength bands are changed, the more subtle feature can be resolved. Instead of redistributing wavelength bands, they can also be rescaled in intensity to reduce cross talk. Down scaling of large input signals with respect to the smaller input signals has the further advantage that the dynamic range of the electronic amplifiers can be chosen in a more optimum way, such that the total dynamic range of the system can be improved.

According to a modification of the embodiment, high-frequency modulation comprising frequencies in the range above 50 MHz is imposed on the beam 11 of spectrally encoded broad-band light. Such a high-frequency modulation can advantageously be utilized to extract additional optical properties from the material, such as optical scattering coefficients (in the case of photon-density-wave analysis) and/or fluorescence lifetime coefficients.

Although an embodiment has been described in which multiple photodetectors are provided, spectroscopy in the region of the turbid medium in front of the tip portion can already be realized by provision of one photodetector in the region of the shaft portion. Instead of at least one optical fiber in combination with a spectrometer for spectroscopy as in the prior art, only a cost-efficient photodetector and an electrical connection to the demodulation and analysis unit 32 are required. According to the proposed realization, the optical spectrum of light that has been scattered immediately in front of the sharp needle tip is acquired with a photodetector and without requiring a spectrometer.

With the proposed realization, information about the microstructure and the molecular composition of the turbid medium (e.g. tissue in the described case of a biopsy needle) immediately in front of the sharp tip portion 22 can be obtained.

With regard to a realization in which a two- or more-dimensional image of the turbid medium in the region of the tip portion is reconstructed the following holds: The more photodetectors are provided in the region of the shaft portion, the better the image can be reconstructed. However, the costs for adding an additional spectroscopic detector will only be the costs of adding an additional photodetector and corresponding electric wiring. This is a particular advantage compared to a solution in which spectroscopic analysis is realized via an optical fiber and a spectrometer.

Since the at least one photodetector is provided directly in the region of the shaft portion 21 which is inserted into the turbid medium (e.g. a mammal body), the problems of small numerical apertures (leading to only a small portion of the scattered light being detectable) which are inherent with coupling of scattered light into optical fibers are overcome.

Although it has been described with regard to the embodiment that the photodetectors 27a, 27b, 27c are embedded in the material of the shaft portion 21, the invention is not restricted to this. For example, a plurality of photodetectors can be provided on a flexible foil which is wrapped around and attached to the shaft portion 21.

Although it has been described with respect to the embodiment that the light guiding structure 23 is arranged in the core element 31 (e.g. formed by a mandrin), it is also possible to arrange the light guiding structure in the material of the shank portion 21.

Although it has been described thus far that the at least one photodetector is arranged at a position on the outer circumference of the shaft portion 21, it is, for instance, also possible to arrange at least one photodetector within the core element 31.

In a realization in which at least two photodetectors 27a, 27b, 27c are provided, it is further possible to perform differential spectroscopy in which the signal of one photodetector is used as a reference for signals corresponding to another photodetector. Differential spectroscopy processing is e.g. described by Amelink and Sterenborg in “Measurement of the local optical properties of turbid media using differential pathlength spectroscopy”, Appl. Opt. 43, 2004, 3048-3054.

Although an embodiment has been described in which the light generating unit 80 is provided in a part of the optical examination device 10 which remains outside the turbid medium, other realizations are also possible. For example, a light generating unit can also be arranged inside the shank portion 21. For example, a small broad-band light source in form of a miniature white LED (which are e.g. being sold by Lumileds® or Nichia® or InfiniLED®) can be provided in the shaft portion 21. Frequency modulation of this light source can, for instance, be performed by means of a small, low-finesse Fabry-Perot element with the length of the cavity varied rapidly in time. Further details about this type of modulation are disclosed by Peng et al. in “Fourier transform emission lifetime spectrometer”, Opt. Lett. 32(4), 2007, 421-423.

As an alternative, the light generating unit may comprise a plurality of light sources which are adapted to emit different bands of wavelengths. The different light sources can be modulated with different characteristics, e.g. at different frequencies. This can, for example, be achieved by independently modulating the power delivered to the respective light sources over time. Similar to the modification described above, the plurality of light sources can be arranged in the shank portion 21.

Although the application of the present invention to a biopsy needle has been described with regard to the embodiments, the invention is not restricted to this and it can also be applied to other medical devices such as catheters or endoscopes. It has been found that the combination of optical sensing and catheters can be clinically valuable in many contexts. The present invention offers a significant simplification in design and improvements in detection sensitivity.

Thus, an optical examination device has been described which is well-suited for a plurality of applications, in particular medical applications. In particular, it can be used in the field of needle biopsy guidance to avoid damage of key structures such as nerves and blood vessels. It can be used for needle-based characterization of tissues within the needle path, such as for the detection of blood vessels and/or nerves and/or for the differentiation between fluid and blood-filled cysts, for example. Further, the optical examination device can e.g. be used to monitor brain tissue, blood vessels, and/or blood flow in the case of needle insertion in the brain.

With regard to catheter applications, the optical examination device can, for instance, be used to characterize plaque in arteries. With regard to endoscope applications, it can e.g. be used to obtain spectroscopic information from tissue outside the shaft of an endoscope and/or from tissue which is visible in the endoscope image.

Although only medical applications of the optical examination device have been described as embodiments, non-medical applications such as optically examining food for testing freshness, quality and content are also possible. For example, the optical examination device can be used for examining the water and/or fat content of food such as butters, oils and spread (e.g. peanut butter), for examining alcohol (ethanol) content, and/or for examining the freshness of e.g. dairy produce.

1. Optical examination device (10) adapted to be at least partially inserted into a turbid medium, the optical examination device comprising a shaft portion (21) adapted to be inserted into the turbid medium, the shaft portion (21) comprising a tip portion (22) adapted to be the foremost portion during insertion into the turbid medium, wherein
at least one light source device adapted to emit a beam (11) of broad-band light is provided in a region of the shaft portion (21) adapted to be inserted into the turbid medium, the beam (11) of broad-band light comprising different wavelength bands (2a, 2b, . . . , 2n) which are differently modulated; and
at least one photodetector (27a, 27b, 27c) for detecting broad-band light is provided in the region adapted to be inserted into the turbid medium of the shaft portion (21).

2. Optical examination device according to claim 1, characterized in that the at least one photodetector (27a, 27b, 27c) is electrically connected to a portion of the optical examination device adapted to remain outside the turbid medium.

3. Optical examination device according to claim 1, characterized in that the at least one photodetector (27a, 27b, 27c) is a photodiode.

4. Optical examination device according to claim 1, characterized in that the shaft portion (21) is provided with a plurality of photodetectors (27a, 27b, 27c) arranged at different positions relative to the shaft portion (21).

5. Optical examination device according to claim 1, characterized in that the optical examination device (10) comprises a demodulation and analysis unit (32) adapted to perform a spectral analysis of a signal received from the at least one photodetector (27a, 27b, 27c).

6. Optical examination device according to claim 1, characterized in that the demodulation and analysis unit (32) is adapted to perform spectral analysis of signals from a plurality of photodetectors (27a, 27b, 27c) and additionally exploit information about respective positions of the plurality of photodetectors (27a, 27b, 27c).

7. Optical examination device according to claim 1, characterized in that the demodulation and analysis unit (32) is adapted to reconstruct a multi-dimensional image of a region of interest of the turbid medium.

8. Optical examination device according to claim 1, characterized in that the shaft portion (21) forms at least a part of a biopsy needle, of a catheter, or of an endoscope.

9. Optical examination device according to claim 1, characterized in that the at least one light source device is formed by the end of a light guiding structure (23) connected to a light generating unit (80) which is adapted to provide the beam (11) of broad-band light.

10. Optical examination device according to claim 9, characterized in that the light guiding structure (23) is arranged in the material of the shaft portion (21) or in a core element (31) which is adapted to be placed in a hollow channel (30) inside the shaft portion.

11. Optical examination device according to claim 1, characterized in that the at least one photodetector (27a, 27b, 27c) is embedded in the material of the shaft portion (21).

12. Optical examination device according to claim 1, characterized in that the optical examination device (10) is adapted such that a high-frequency modulation in a frequency range above 50 MHz is imposed on the beam (11) of broad-band light.

13. Optical examination device according to claim 1, characterized in that the optical examination device is a medical device adapted to be at least partially inserted into a mammal body.

14. Optical examination device according to claim 1, characterized in that the at least one light source device is provided in the region of the tip portion (22).
